(19) United States
(12) Patent Application Publication
(10) Pub. No.: US 2026/0259141 A1
(43) Pub. Date: Sep. 3, 2026

(54) METHODS FOR ANTIGEN RETRIEVAL OF A FIXED SAMPLE FOR MULTIPLE APPLICATIONS

(71) Applicant: RESOLVE BIOSCIENCES GMBH, Monheim am Rhein (DE)

(72) Inventors: Treewut Rassamegevanon, Monheim (DE); Andreas Meier, Düsseldorf (DE); Christian Korfhage, Langenfeld (DE)

(21) Appl. No.: 19/163,728

(22) PCT Filed: Feb. 29, 2024
(86) PCT No.: PCT/EP2024/055235

§ 371 Date: Sep. 9, 2025

Related U.S. Application Data
(60) Provisional application No. 63/453,284, filed on Mar. 20, 2023.
(60) Provisional application No. 63/456,613, filed on Apr. 3, 2023.

(51) Int. Cl. G01N 21/64; G01N 1/30; G01N 33/554; G01N 35/00

(52) U.S. Cl. CPC G01N 21/6428; G01N 1/30; G01N 21/6456; G01N 33/554; G01N 35/0099; G01N 2001/305; G01N 2021/6439; G01N 2333/902

(57) ABSTRACT

The technology provided herein relates to methods for antigen retrieval of a fixed sample, in particular of frozen-fixed tissue samples by using a two-step, demasking approach, in which biological samples are treated at low-temperature (50-75° C.) with different buffers.

### CROSS REFERENCE TO RELATED APPLICATIONS

This is a US national phase application under 35 U.S.C. § 371 of international application no. PCT/EP2024/055235, filed 29 Feb. 2024, which claims priority to U.S. application No. 63/453,284 filed 20 Mar. 2023 and U.S. 63/456,613 filed Apr. 3, 2023; each application referenced in this paragraph is incorporated herein by reference in its entirety.

### FIELD OF THE DISCLOSURE

The technology provided herein relates to methods for antigen retrieval of a fixed sample, in particular of frozen-fixed tissue samples by using a two-step, demasking approach, in which biological samples are treated at low-temperature (50-75° C.) with different buffers.

### BACKGROUND

In the fields of histology, pathology, and cell biology, fixation is the preservation of biological tissues from decay due to autolysis or putrefaction. It terminates any ongoing biochemical reactions and may also increase the treated tissues' mechanical strength or stability. Tissue fixation is a critical step in the preparation of histological sections, its broad objective being to preserve cells and tissue components and to do this in such a way as to allow for the preparation of thin, stained sections. This allows the investigation of the tissues' structure, which is determined by the shapes and sizes of such macromolecules (in and around cells) as proteins and nucleic acids.

The nature of tissue processing requires that the samples be “fixed” prior to embedding in paraffin and micro-sectioning on a microtome to produce tissue sections suitable for immunostaining. During this process, proteins are preserved using a formaldehyde treatment that produces chemical cross-linking which preserves the cellular features of the tissue. Formaldehyde preserves or fixes tissue or cells predominantly by cross-linking primary amine groups in proteins with other nearby nitrogen atoms in protein or DNA through a —CH2— linkage. The process of tissue fixation however, frequently masks antigens on specific proteins for which detection is desirable for diagnostic and prognostic purposes. Typically procedures are optimized for detection of individual target molecules, and when needed, serial sections are processed in a different manner for the detection of additional targets molecules. With advances in the ability to detect multiple antigens in a single sample, there needs to be a uniform tissue processing that is compatible with the detection of multiple proteins. S Yamashita et al in J Histochem Cytochem 53, 13-21 (2005) discloses heating of formaldehyde-fixed samples in a series of two Tris-HCl buffers. US2004/035364 discloses antigen retrieval by heating in low pH buffers, while S R Shi et al in J Histochem Cytochem 39, 741-748 discloses microwave heating of formaldehyde-fixed samples in different antigen retrieval solutions.

One technique based on Gerdes et al. (U.S. Pat. No. 8,067,241) is a method of antigen retrieval of a formaldehyde-fixed tissue sample comprising the step of incubating the formaldehyde-fixed tissue sample in a first antigen retrieval solution, transferring the tissue sample to a second antigen retrieval solution, and incubating the tissue sample in the second antigen retrieval solution.

The system is designed for demasking epitopes of interest at a temperature of greater than 90° C. on formalin-fixed, paraffin embedded tissues. The formalin-fixed, paraffin embedded tissues are incubated in the first antigen retrieval solution (pH 5-7). The tissue samples are then transferred without additional treatment to the second antigen retrieval buffer solution (pH 7.5-11). The first and the second antigen retrieval buffer may be switched. The incubation is performed for longer than 10 minutes, preferable for a period of approximately 20 minutes.

The methods for detection of multiple analytes known in the art have numerous disadvantages. Against this background, it is an object underlying the present disclosure to provide a method by means of which the disadvantages of the prior art methods can be reduced or even avoided.

### SUMMARY OF THE DISCLOSURE

The present disclosure pertains to novel low-temperature sample treatment procedures for in-situ detection of biomolecules in fixed biological samples like formaldehyde fixed biological samples.

Crosslinking fixation can change biochemistry of biomolecules, making them unable to bind to the specific binding molecules. This is caused by intra-and inter crosslinking of biomolecules by formaldehyde, changing the conformation of biomolecules, creating steric hinderance between biomolecules and the specific binding molecules, masking the binding site for the specific binding molecules. A two-step, demasking approach, in which biological samples are treated at low-temperature (50-75° C.) with a light acidic buffer (pH 6.5-7) and a light basic buffer (pH 8-10), consecutively, for certain period (0.5-2 h; each step), reduces the masking of biomolecules. Thereby, the ability to form a specific binding between molecules is restored. Heating may occur using a water bath, hot plate, microwave, or thermal cycler, to provide uniform heating to tissue samples immersed in the demasking solution. The presence of biomolecules in biological samples can be identified via histological techniques as in situ hybridization and immunohistochemistry, facilitating a multi-omics analysis. These two histological techniques can be performed on the same slide in a consecutive manner upon applying the purposed demasking method.

However, this approach is not limited to biological samples fixed in crosslinking fixatives but also applicable for biological samples fixed in coagulant fixatives e.g., alcohol, and acetone, biological samples that were fixed in formaldehyde and embedded in paraffin (FFPE), and frozen fixed biological samples.

In a first aspect, the present disclosure pertains to a method of antigen retrieval of a fixed sample comprising the steps of:
a) incubating the fixed sample in a first antigen retrieval solution at a temperature between 50 to 75° C.;
b) transferring the fixed sample to a second antigen retrieval solution; and
c) incubating the fixed sample in the second antigen retrieval solution at a temperature between about 50 to 75° C.,
wherein the first antigen retrieval solution comprises a buffer solution having a pH range of between about 6.5 and 7 and the second antigen retrieval solution comprises a buffer solution having a pH range of between about 8 and 10; or
the first antigen retrieval solution comprises a buffer solution having a pH range of between about 8 and 10 and the second antigen retrieval solution comprises a buffer solution having a pH range of between about 6.5 and 7.

Before the disclosure is described in detail, it is to be understood that this disclosure is not limited to the particular component parts of the steps of the methods described. It is also to be understood that the terminology used herein is for purposes of describing particular embodiments only, and is not intended to be limiting. It must be noted that, as used in the specification and the appended claims, the singular forms “a,” “an” and “the” include singular and/or plural referents unless the context clearly dictates otherwise. It is moreover to be understood that, in case parameter ranges are given which are delimited by numeric values, the ranges are deemed to include these limitation values.

### BRIEF DESCRIPTION OF THE DRAWINGS

FIG. 1 is an overview of embodiments of the the two-step demasking process on fixed tissue for multi-omics analysis.

FIG. 2 shows cryosections of murine kidney tissues were probed for Lysozyme, MDM2, and p21. Rabbit primary antibody against lysozyme (BioLegend, #860001) and mouse primary antibody against MDM2 (Santa Cruz, sc-965) and p21 (Origene, AM09100PU-T) were incubated on the tissues, which were pretreated with the two-step demasking method. Alexa Flour 647 conjugated to goat anti-rabbit IgG (Abcam, ab150083) were added after the primary antibody incubation. Images were captured with ZEISS Celldiscoverer 7 using a Apochromat 5X/0.35 objective and 2X Tubelens.

FIG. 3 shows a further cryosection of a tissue from a colorectal xenograft model by using the method for antigen retrieval according to the present disclosure.

### DETAILED DESCRIPTION OF THE DISCLOSURE

The present disclosure features methods for antigen retrieval in a fixed sample, which conducts in a sequential manner. The method is suitable for fixed tissues (frozen-fixed or formalin fixed) and stored at −80° C. or by embedding tissues in paraffin.

Frozen fixed tissues can be post-fixed with coagulant fixatives (alcohol or acetone) or crosslinking fixatives (formaldehyde). As mentioned above, the present disclosure pertains to a two-step, demasking approach, in which biological samples are treated at low-temperature (50-75° C.) with a light acidic buffer (pH 6.5-7) and a light basic buffer (pH 8-10), consecutively, for certain period (0.5 −2 h; each step), which reduces the masking of biomolecules. Thereby, the ability to form a specific binding between molecules is restored. Heating may occur using a water bath, hot plate, microwave, or thermal cycler, to provide uniform heating to tissue samples immersed in the demasking solution.

The presence of biomolecules in biological samples can be identified via histological techniques as in situ hybridization and immunohistochemistry, facilitating a multi-omics analysis. These two histological techniques can be performed on the same slide in a consecutive manner upon applying the purposed demasking method.

This approach is not limited to biological samples fixed in crosslinking fixatives but also applicable for biological samples fixed in coagulant fixatives e.g., alcohol, and acetone, biological samples that were fixed in formaldehyde and embedded in paraffin (FFPE), and frozen fixed biological samples.

Surprisingely it was found that in the antigen retrieval process of the prior art (U.S. Pat. No. 8,067,241) the temperature is too high, which is not applicable for frozen-fixed tissues. The incubation in high temperature may damage tissue structure and morphology or leads to tissue detachment from the holder. Furthermore this technique is applicable to solely tissues that are formaldehyde fixed and embedded in paraffin. The high temperature treatment (over 80° C.) is further not suitable for instrument used for spatial biology analysis platforms and in particular this technique is not suitable if in-situ hybridization and immunofluorescence are performed on the same tissue samples.

The analysis and detection of small quantities of analytes like RNA or proteins in biological and non-biological samples has become a routine practice in the clinical and analytical environment. Numerous analytical methods have been established for this purpose. Some of them use encoding techniques assigning a particular readable code to a specific first analyte which differs from a code assigned to a specific second analyte.

One of the prior art techniques in this field is the so-called ‘single molecule fluorescence in situ hybridization’ (smFISH) essentially developed to detect mRNA molecules in a sample. In Lubeck et al. (2014), Single-cell in situ RNA profiling by sequential hybridization, Nat. Methods 11(4), p. 360-361, the mRNAs of interest are detected via specific directly labeled probe sets. After one round of hybridization and detection, the set of mRNA specific probes is eluted from the mRNAs and the same set of probes with other (or the same) fluorescent labels is used in the next round of hybridization and imaging to generate gene specific color-code schemes over several rounds. The technology needs several differently tagged probe sets per transcript and needs to denature these probe sets after every detection round.

A further development of this technology does not use directly labeled probe sets. Instead, the oligonucleotides of the probe sets provide nucleic acid sequences that serve as initiator for hybridization chain reactions (HCR), a technology that enables signal amplification; see Shah et al. (2016), In situ transcription profiling of single cells reveals spatial organization of cells in the mouse hippocampus, Neuron 92(2), p. 342-357.

Immunohistochemistry (IHC) refers to a processes of detecting, localizing, and/or quantifying antigens, such as a protein, in a biological sample using antibodies specific to the particular antigens. IHC provides the substantial advantage of identifying exactly where a particular protein is located within the tissue sample. It is also an effective way to examine the tissues themselves. In situ hybridization (ISH) refers to the process of detecting, localizing, and quantifying nucleic acids. Both IHC and ISH can be performed on various biological samples, such as tissue (e.g. fresh frozen, formalin fixed, paraffin embedded) and cytological samples. Recognition of the targets can be detected using various labels (e.g., chromogenic, fluorescent, luminescent, radiometric), irrespective of whether the target is a nucleic acid or an antigen. To robustly detect, locate, and quantify targets in a clinical setting, amplification of the recognition event is desirable as the ability to confidently detect cellular markers of low abundance becomes increasingly important for diagnostic purposes. For example, depositing at the marker's site hundreds or thousands of label molecules in response to a single antigen detection event enhances, through amplification, the ability to detect that recognition event.

According to the present disclosure an “antigen” is the subject to be specifically detected as being present or absent in a sample and, in case of its presence, to encode it. It can be any kind of entity, including a protein, polypeptide, protein or a nucleic acid molecule (e.g. RNA, PNA or DNA) of interest. The analyte provides at least one site for specific binding with an antibody, antibody fragment and/or an aptamer.

“Antigen” refers in particular to a substance that may bind an antibody, anantibody fragment and/or an aptamer. Antigens may be endogenous whereby they are generated within the cell as a result of normal or abnormal cell metabolism, or because of viral or intracellular bacterial infections. Endogenous antigens include xenogenic (heterologous), autologous and idiotypic or allogenic (homologous) antigens. Antigens may also be tumor-specific antigens or presented by tumor cells. In this case, they are called tumor-specific antigens (TSAs) and, in general, result from a tumor-specific mutation. Antigens may also be tumor-associated antigens (TAAs), which are presented by tumor cells and normal cells. Antigen also includes CD antigens, which refers any of a number of cell-surface markers expressed by leukocytes and can be used to distinguish cell lineages or developmental stages. Such markers can be identified by specific monoclonal antibodies and are numbered by their cluster of differentiation.

In particular, the antigen may be detected or bound by an antibody, an antibody fragment, an aptamer or a nucleic acid that specifically interacts and binds to the analyte, wherein in particular the antibody fragment is selected from the group consisting of a Fab, scFv, single domain, bis scFv, Fab2, Fab3, minibody, diabody, triplebody, tetrabody and tandab.

“FISH” and “CISH” refer to fluorescent in situ hybridization and chromagenic in situ hybridization respectfully. FISH is a cytogenetic technique used to detect and localize the presence or absence of specific DNA sequences on chromosomes or RNA sequences at transcription sites as well as in other parts of the cell. FISH uses fluorescent probes that bind to only those parts of the chromosome with which they show a high degree of sequence similarity. CISH allows detection of gene amplification, chromosome translocations and chromosome number using conventional enzymatic reactions under the brightfield microscope on formalin-fixed, paraffin-embedded (FFPE) tissues. “Immunostaining” refers to an antibody-based and/or aptamer-based method to detect a specific protein in a sample. Immunostainging includes both immunocyctochemical staining and immunhistochemical staining. Immunocyctochemical (ICC) staining refers to a techniue that uses antibodies which target antigenson the cells. This may be performed to determine the presence of certain diseases, for example, types of cancer. Immunohistochemical (IHC) staining refers to the staining and localization of antigens in tissue sections by the use of labeled antibodies as specific reagents through antigen-antibody interactions that are visualized by a marker such as fluorophores, reacted enzyme substrates, radioactive element or colloidal gold.

A “sample” as referred to herein is a composition in liquid or solid form suspected of comprising the analytes to be encoded. In particular, the sample is a biological sample, preferably comprising biological tissue, further preferably comprising biological cells and/or extracts and/or part of cells. For example, the cell is a prokaryotic cell or a eukaryotic cell, in particular a mammalian cell, in particular a human cell. In some embodiments, the biological tissue, biological cells, extracts and/or part of cells are fixed. In particular, the analytes are fixed in a permeabilized sample, such as a cell-containing sample.

As used in the present disclosure, “cell”, “cell line”, and “cell culture” can be used interchangeably and all such designations include progeny. Thus, the words “transformants” or “transformed cells” include the primary subject cell and cultures derived therefrom without regard for the number of transfers. It is also understood that all progeny may not be precisely identical in DNA content, due to deliberate or inadvertent mutations. Mutant progeny that have the same functionality as screened for in the originally transformed cell are included.

In some advantageous embodiments, the fixed sample is a biological sample, preferably comprising biological tissue from human, animals, or plants, further preferably comprising biological cells and/or extracts and/or part of cells. In particular, the cell is a prokaryotic cell or a eukaryotic cell, an animal cell, a plant cell, in particular a cell from vertebrate cells, in particular a mammalian cell, in particular a human cell.

The fixation of the biological sample can be carried out with all fixatives known to the person skilled in the art, in particular with acids, alcohols, ketones or other organic substances, such as glutaraldehyde or formaldehyde, with formaldehyde-fixed biological samples and with an aqueous formaldehyde solution, containing 1 to 35 wt.-%, preferably 2 to 10 wt.-% formaldehyde. According to an embodiment of the method according to the present disclosure, a biological sample fixed in formaldehyde and embedded in paraffin is used as a fixed sample. Such samples are commonly referred to as FFPE samples. Such a FFPE sample is preferably prepared by first dewatering a formalin-fixed biological sample, preferably by means of an ascending alcohol series (ie a series of water / alcohol mixtures with increasing amounts), the alcohol concentration, ultimately pure alcohol is added). Cis to Cs alcohols are particularly preferred as alcohols, and ethanol, methanol and isopropanol are moreover preferred and ethanol is most preferred. Subsequently, the dehydrated sample is dipped in liquid paraffin, which is cured after the sample has been sufficiently penetrated by the paraffin. By means of suitable cutting devices, for example by means of a microtome, tissue sections can then be produced from the paraffin block, wherein the thickness of these sections for light microscopic examination is usually about 5 to 20 μm.

In an advategeous embodiment, the fixed sample is a frozen-fixed tissue sample. For example, the fixed sample is a cryosection of a tissue. Cryosections are rapidly and relatively easily prepared prior to fixation, and they provide a good system for visualizing fine details of the cell. Although cryosections are physically less stable than paraffin-or resin-embedded sections, they are generally superior for the preservation of antigenicity and therefore the detection of antigens by microscopy. The preparation of cryosections does not involve the dehydration steps typical of other sectioning methods, and, furthermore, sectioning, labeling, and observation of specimens can usually be carried out in one day. In general, the sample is frozen quickly in either isopentane or liquid nitrogen. (Small samples such as cells and small tissues may be mixed in a slurry of an inert support medium such as optimal cutting temperature [OCT] compound before freezing). Rapid freezing reduces ice crystal formation and minimizes morphological damage. Frozen sections may be used for a variety of procedures, including immunochemistry, enzymatic detection, and in situ hybridization. In some advategeous embodiments, the fixed-sample is a frozen formaldehyde-fixed biological sample, in particular a formaldehyde-fixed cryosection of a tissue.

In some advategeous embodiments, the sample is stored before the antigen retrieval/demasking of the biomolecules at −80° C.

In some embodiments, the fixed sample is a formaldehyde-fixed biological sample, in particular the formaldehyde-fixed biological sample is a formaldehyde-fixed and paraffin-embedded biological sample. In some embodiments, the paraffin is at least partially removed prior to contacting the sample with the first first antigen retrieval solution (first buffer). In advantageous examples, the sample is fixed in formaldehyde and embedded in paraffin (FFPE).

In advantageous examples, the sample is a frozen fixed tissue sample like a cryosection of a tissue and post-fixed with coagulant fixatives like alcohol or acetone and/or with crosslinking fixatives like formaldehyde.

In the “incubation” steps as understood herein the respective moieties or subjects such as samples or probes, are brought into contact with each other under conditions described in the present disclosure or are well known to the skilled person allowing a chemical reaction like the demasking of the antigens in the fixed samples, e.g. pH, temperature, salt conditions etc. Such steps may therefore, be preferably carried out in a liquid environment such as a buffer system.

As mentioned above, the present disclosure pertains also to a method of demasking biomolecules in a fixed sample, comprising the steps of:
a) incubating the fixed sample in a first buffer solution at a temperature between 50 to 75° C.;
b) transferring the fixed sample to a second buffer solution; and
c) incubating the fixed sample in the second buffer solution at a temperature between about 50 to 75° C., wherein the first buffer solution has a pH range of between about 5 and 7 and the second buffer solution has a pH range of between about 8 and 10; or
the first buffer solution has a pH range of between about 8 and 10 and the second antigen buffer solution has a pH range of between about 5 and 7.

In an embodiment of the disclosure the biological tissue and/or biological cells are fixed. Techniques for fixing cells are known to those of ordinary skill in the art. As non-limiting examples, a cell may be fixed using chemicals such as formaldehyde, paraformaldehyde, glutaraldehyde, ethanol, methanol, acetone, acetic acid, or the like.

The invention includes embodiments that relate generally to methods applicable in analytical, diagnostic, or prognostic applications such as analyte detection, histochemistry, immunostaining, immunohistochemistry, immunocytochemistry or immunofluorescence. In some embodiments, the methods disclosed herein may be particularly applicable in immunohistochemistry and immunocytochemistry.

In accordance with one embodiment, a method is described in which a tissue section derived from pathological sampling is processed prior to protein detection for biomarker assessment. In one embodiment, the method comprises a two-step procedure that is applicable to multiple protein antigens and may provide for high level of antigen retrieval. In certain embodiments, this allows for multiplexing diagnosis of clinically relevant samples.

In some embodiments, a tissue sample includes tissue sections from healthy or diseased tissues (e.g., tissue sections from colon, breast tissue, prostate). A tissue sample may include a single part or piece of a tissue section, for example, a thin slice of tissue or cells cut from a tissue section. In some embodiments, the same section of tissue sample may be analyzed at both morphological and molecular levels.

In some embodiments, the tissue sample may be first fixed and then dehydrated through an ascending series of alcohols, infiltrated and embedded with paraffin or other sectioning media so that the tissue sample may be sectioned. In an alternative embodiment, a tissue sample may be sectioned and subsequently fixed. In some embodiments, the tissue sample may be embedded and processed in paraffin. Examples of paraffin that may be used include, but are not limited to, Paraplast, Broloid, and Tissuecan. Once the tissue sample is embedded, the sample may be sectioned by a microtome into sections. The thickness of the sections may vary based on the type of tissue and analysis. In certain embodiments the sections may have a preferred thickness in a range of from about two microns to about five microns.

Once sectioned, the sections may be attached to slides using adhesives. Examples of slide adhesives may include, but are not limited to, silane, gelatin, poly-L-lysine. In embodiments, if paraffin is used as the embedding material, the tissue sections may be deparaffinized and rehydrated in water. The tissue sections may be deparaffinized, for example, by using organic agents (such as, xylenes or gradually descending series of alcohols).

In other embodiments, a formaldehyde fixed tissue sample may be adhered to a solid support in order to allow for its analysis, transfer and movement during the preparation and imaging processes. The tissue sample may be immobilized on the solid support by physical adsorption, by covalent bond formation, or by combinations thereof. A solid support may include a polymeric, a glass, or a metallic material. Examples of solid supports include a membrane, a microtiter plate, a bead, a filter, a test strip, a slide, a cover slip, and a test tube.

In one embodiment a method is described in which a fixed sample like a formaldehyde fixed tissue sample is contacted with a first antigen retrieval solution and heated to a temperature of 50 to 75° C. for a period of at least 0.5 h, in particular for about 0.5 to about 2 hours (h), more preferable to a temperature of about 70° C. and in particular for a period of about 0.5 h., more preferable to a temperature of 70° C. and in particular for a period of 0.5 h.

Heating may occur using a pressure cooker, oven, stove, autoclave, water bath, hot plate, microwave, or steam heater, to provide uniform heating to the tissue sample immersed in the antigen retrieval solution.

The tissue sample is then transferred without additional treatment, but in particular after resting, to a second antigen retrieval solution that was pre-heated to a temperature of 50 to 75° C. for a period of at least 0.5 h, in particular for about 0.5 to about 2 hours (h), more preferable to a temperature of about 70° C. and in particular for a period of about 0.5 h.

Pre-heating may occur using a pressure cooker, autoclave, water bath, hot plate, microwave, steam heat or a combination thereof and can be performed at the time of heating the first antigen retrieval solution. Preferably the incubation of the sample in the second antigen retrieval solution occurs at atmospheric pressure, and by immersion only in the hot solution. This may prevent tissue damage.

In one embodiment, the first antigen retrieval solution is a buffer solution having a pH range between about 5 and about 7, in particular between about 6 and about 7. The first antigen retrieval solution may be a commonly used buffer solution used to maintain pH in the range of slightly acidic to neutral. In certain embodiments the buffer may comprise citric acid, potassium dihydrogen phosphate, diethyl barbituric acid, piperazine-N,N′-bis(2-ethanesulfonic acid), dimethylarsinic acid, 2-(N-morpholino)ethanesulfonic acid, or a combinations thereof. In other embodiments, the buffer solution may be a citric acid sodium phosphate buffer solution.

In one embodiment, the second antigen retrieval solution is a buffer solution having an alkaline pH in the range of about 8 to about 10. The second antigen retrieval solution may be a commonly used buffer solution use to maintain pH in a slightly alkaline range. In certain embodiments, the buffer solution may be comprised of tris(hydroxymethyl)methylamine (TRIS), boric acid, sodium tetraborate, 2-(N-morpholino)ethanesulfonic acid (TAPS), N,N-bis(2-hydroxyethyl)glycine(Bicine), N-tris(hydroxymethyl)methylglycine (Tricine), 4-2-hydroxyethyl-1-piperazineethanesulfonic acid (HEPES), 2-{[tris(hydroxymethyl)methyl]amino}ethanesulfonic acid (TES), or a combination thereof. In another embodiment, the buffer solution may be a TRIS-HCl buffer having a pH of approximately 10 at elevated temperatures.

In particular, the first antigen retrieval solution is defined in general as a buffer for the first demasking step and may havea pH value in a range of 5-7 or 8-10. In particular, the second antigen retrieval solution is defined in general as a buffer for the second demasking step. Buffer B may have pH value in a range of 5-7 or 8-10.

It should be appreciated that in other embodiments, the first antigen retrieval solution may be a buffer solution in the range of about 8 to about 10 and the second antigen retrieval solution may be a buffer solution in the range of about 5 to about 7, in particular of about 6 to about 7, in particular between 6.5 and 7.

In summary, the present disclosure pertains to a two-step, demasking approach, in which biological samples are treated at low-temperature (50-75° C.) with a light acidic buffer (pH 5-7) and a light basic buffer (pH 8-10), consecutively, for certain period (0.5-2 h; each step), reduces the masking of biomolecules. Thereby, the ability to form a specific binding between molecules is restored. Heating may occur using a water bath, oven, stove, hot plate, microwave, or thermal cycler, to provide uniform heating to tissue samples immersed in the demasking solution. The presence of biomolecules in biological samples can be identified via histological techniques as in situ hybridization and immunohistochemistry, facilitating a multi-omics analysis. These two histological techniques can be performed on the same slide in a consecutive manner upon applying the purposed demasking method.

The antigen retrieved by exposure to the first and second antigen retrieval solutions may be more susceptible to immunostaining to allow for both analytical and functional morphology studies. Immunostaining includes both immunohistochemical (IHC) staining and immunocytochemical (ICC) staining. In certain embodiments improvement may include increased intensity of positive staining and decrease background staining.

Therefore, the first or the second antigen retrieval solution may comprise citric acid, Trisodium citrate, Phosphate, 2-(N-Morpholino)ethanesulfonic acid hydrate, Bis(2-hydroxyethyl)amino-tris(hydroxymethyl)methane, N-(2-Acetamido)iminodiacetic acid, N-(Carbamoylmethyl)iminodiacetic acid, N-(2-Acetamido)-2-aminoethanesulfonic acid, 1,4-Piperazinediethanesulfonic acid, 3-Morpholino-2-hydroxypropanesulfonic acid, 1,3-Bis[tris(hydroxymethyl)methylamino]propane, 3-(N-Morpholino)propanesulfonic acid, N,N-Bis(2-hydroxyethyl)-2-aminoethanesulfonic acid, Disodium hydrogen phosphate, Monosodium phosphate, Imidazole, 2-[(2-Hydroxy-1,1-bis(hydroxymethyl)ethyl)amino]ethanesulfonic acid, 4-(2-Hydroxyethyl)piperazine-1-ethanesulfonic acid, or Tris(hydroxymethyl)aminomethane. 4-(N-Morpholino)butanesulfonic acid, Piperazine-1,4- bis(2-hydroxypropanesulfonic acid) dihydrate, 4-(2-Hydroxyethyl)-1-piperazinepropanesulfonic acid, N-[Tris(hydroxymethyl)methyl]glycine, Diglycine, N,N-Bis(2-hydroxyethyl)glycine, N-(2-Hydroxyethyl)piperazine-N'-(4-butanesulfonic acid), N-[Tris(hydroxymethyl)methyl]-3-aminopropanesulfonic acid, 2-Amino-2-methyl-1,3-propanediol, N-(1,1-Dimethyl-2-hydroxyethyl)-3-amino-2-hydroxypropanesulfonic acid, 2-(Cyclohexylamino)ethanesulfonic acid, 3-(Cyclohexylamino)-2-hydroxy-1-propanesulfonic acid, Boric Acid, Sodium tetraborate, Ethylenediaminetetraacetic acid, Sodium bicarbonate, Sodium carbonate and Non-ionic detergent.

In certain embodiments, after the application of the second antigen retrieval solution, immunostaining of the sample may occur. An antibody solution (e.g. a probe) may be contacted with the tissue section for a sufficient period of time and under conditions suitable for binding of the labeled-antibody to the antigen. Two detection methods may be used: direct or indirect. In a direct detection, a signal generator-labeled primary antibody (e.g., fluorophore-labeled primary antibody) may be incubated with an antigen in the tissue sample, which may be visualized without further antibody interaction. In an indirect detection, an unconjugated primary antibody may be incubated with an antigen and then a labeled secondary antibody may bind to the primary antibody. Signal amplification may occur as several secondary antibodies may react with different epitopes on the primary antibody. In embodiments where the secondary antibody may be conjugated to an enzymatic label, a chromogenic or fluorogenic substrate may be added to provide visualization of the antigen. In some embodiments two or more (at most four) primary antibodies (labeled or unlabeled) may be contacted with the tissue sample. Unlabeled antibodies may be then contacted with the corresponding labeled secondary antibodies. In some embodiments, other methods may be employed for signal enhancement such as the use of a labeled tertiary or quaternary antibody.

In some embodiments after the antigen retrieval process, nucleic acid probes are applied to the sample to perform fluorescent in situ hybridization (FISH) or chromagenic in situ hybridization (CISH).

By this measure the method is further developed to such an extent that the encoded analytes can be detected by any means which is adapted to visualize the signal element. Examples of detectable physical features include e.g. light, chemical reactions, molecular mass, radioactivity, etc.

In some advantageous embodiments, the signal caused by the signal element, therefore in particular the binding of the signal oligonucleotides to the decoding oligonucleotides, interacting with the corresponding analyte probes, bound to the respective analyte is determined by:
(a) Imaging at least a portion of the sample; and/or
(b) Using an optical imaging technique; and/or
(c) Using a fluorescence imaging technique; and/or
(d) Multi-color fluorescence imaging technique; and/or
(e) Super-resolution fluorescence imaging technique.

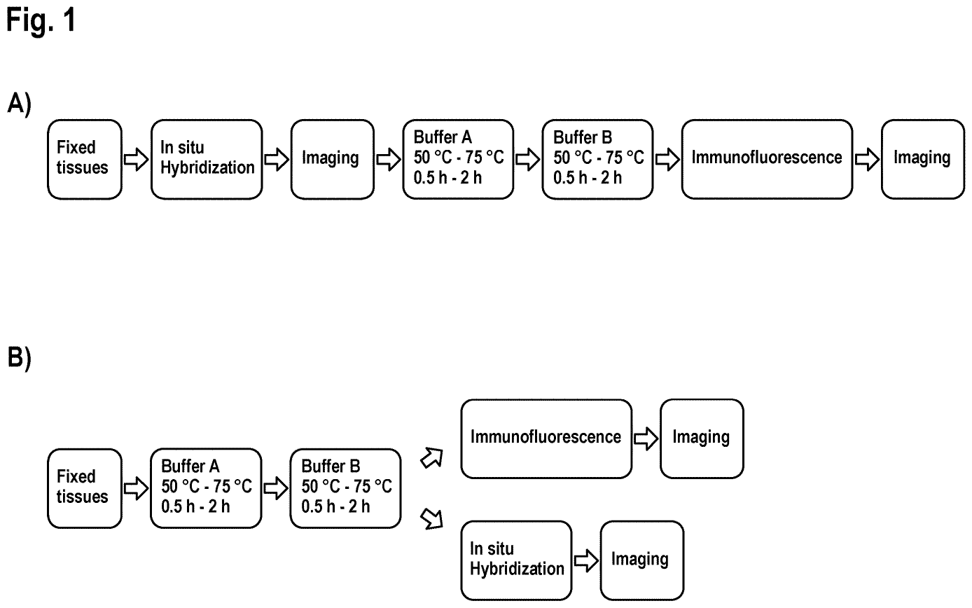

The sequential demasking process may be applied in 2 different manners (see FIG. 1):
A) Post in situ hybridization, prior to immunofluorescence staining.
B) Prior to in situ hybridization or immunofluorescence staining

In general, the two-step demasking method is performed as follows:
1) Fixed tissues, on which either in situ hybridization was analyzed or freshly prepared, are incubated with buffer A at 50-75° C. for 0.5-2 h. A preferential condition is 70° C. for 0.5 h.
2) Tissues are rested and transferred to buffer B and incubated at 50-75° C. for 0.5-2 h. A preferential condition is 70° C. for 0.5 h.
3) Tissues are rested and detection of biomolecules thereafter using immunohistochemical staining or in situ hybridization.

In particular, the methods of the present disclosure may be used to identify regions of interest, in particular in an organ, tissue or single cell.

In some advantageous embodiments, the methods of the present disclosure can be used for image analysis. In this context, the methods of the present disclosure are used to develop cell segmentation algorithms using immunofluorescence images. The methods of the present disclosure could be used to aid the segmentation of cells in a sample, in particular in a tissue. Cell segmentation is a crucial step since errors in identifying cells and their boundaries have a direct impact on the ability to correctly quantify the expression levels of proteins in these cells. For example, information about the levels of proteins in single cells can also be used to answer questions regarding the set of cell types in a sample, the spatial distribution of these types, and the interactions between different cells and cell types in a tissue.

A unique advantage of spatial proteomics is that, unlike prior methods, they do not require specific markers to be selected in advance. Since these studies profile tens of proteins, a marker can be selected post-experiment. Moreover, there is no need to select a single marker. Even if a single marker does not work for all cells, a combination of such markers may be useful for segmenting all cells.

Furthermore, the methods of the present disclosure provide spatial information about the location of the cells being profiled enabling the analysis of cell-cell signaling and cell type organization. By using the methods according to the present disclosure of detecting analytes like biomolecules in a fixed biological sample, up to three analytes like proteins on the surface of cells could be detected thereby visualizing the boundaries of cells. The analyte may be bound by an antibody, an antibody fragment, an aptamer or a nucleic acid that specifically interacts and binds to the analyte, wherein in particular the antibody fragment is selected from the group consisting of a Fab, scFv, single domain, bis scFv, Fab2, Fab3, minibody, diabody, triplebody, tetrabody and tandab.

In particular, the analyte to be detected may be Sodium-potassium ATPase, plasma membrane calcium ATPase (PMCA), Cadherin, CD98, Caveolae, HER2, Beta-Catenin, CAH9, E-Cadherin, c-MYC, Vimentin, among others. The choice of the binding elements like antibodies will vary based on the cell type to be detected. Each tissue has a different composition of cell types. By visualizing the boundaries of cells, a user could by eye or through a computational tool segment cell.

In some further advantageous embodiments, the methods of the present disclosure may be used to establish Al-based image analysis algorithm using immunofluorescence images to identify disorders. Furthermore, the advantageous embodiments the methods of the present disclosure may be used for feature extraction like nucleus morphology, signal homogeneity, signal intensity and/or centroids. For example, the morphology of cells often reflects their tissue-specific function or state. Cells undergo morphological changes to become motile in response to various stimuli, either as part of normal physiology and development, or due to pathologic disorder. In some cancers, epithelial cells lose their inter-cellular connections, and take on protrusive, exploratory morphologies as they transit from static to migratory phenotypes. Recognizing such abnormal cell shapes can aide in properly identifying malignant (as distinct from benign) tumors.

A further application for the methods of the present disclosure is to determine expression of proteins and genes of interest via semi-quantitative approaches. Semi-quantitative immunohistochemistry (IHC) for example is a powerful method for investigating protein expression and localization within tissues.

In some further advantageous embodiments, the methods of the present disclosure may be used for diagnosis and prognosis applications. In particular, the methods of the present disclosure are used for the determination of the distribution of a target protein in the of health and disease organ, tissue and/or cell. Furthermore, the methods of the present disclosure may be used to predict the prognosis of diseases by using disease-related prognostic and/or predictive biomarkers. A variety of factors influence a patient's clinical outcome, including intrinsic characteristics of the patient, disease, or medical condition, and the effects of any treatments that the patient receives. Some of the intrinsic characteristics may be reflected as prognostic biomarkers, i.e., biomarkers used to identify likelihood of a clinical event, disease recurrence or progression in patients who have the disease or medical condition of interest, and others as predictive biomarkers, i.e., biomarkers used to identify individuals who are more likely than similar individuals without the biomarker to experience a favorable or unfavorable effect from exposure to a medical product or an environmental agent.

In some further advantageous embodiments, the methods of the present disclosure may be used to provide disease diagnosis e.g. tumor of unknow origin, neurodegenerative disorders, muscle disorders, brain trauma, infectious diseases. In particular, the methods of the present disclosure are used to predict a therapeutic response. A further application of the methods of the present disclosure is the subclassification of diseases. Classifications of diseases become extremely important in the compilation of statistics on causes of illness (morbidity) and causes of death (mortality).

Furthermore, the methods of the present disclosure may be used to examine gene expression and to study gene regulation in normal and pathological tissues.

In some further advantageous embodiments, the methods of the present disclosure may be used for morphological studies. In particular, the methods of the present disclosure are used for the recognition of tissue structures and/or cell type classifications. In this context, a cell type is a classification used to identify cells that share morphological or phenotypical features. A multicellular organism may contain cells of a number of widely differing and specialized cell types, such as muscle cells and skin cells, that differ both in appearance and function yet have identical genomic sequences. Cells may have the same genotype, but belong to different cell types due to the differential regulation of the genes they contain. Classification of a specific cell type is often done through the use of microscopy (such as those from the cluster of differentiation family that are commonly used for this purpose in immunology).

In some further advantageous embodiments, the methods of the present disclosure may be used for multiomics studies. Multiomics is a biological analysis approach in which the data sets are multiple “omes”, such as the genome, proteome, transcriptome, epigenome, metabolome, and microbiome. By combining these “omes”, scientists can analyze complex biological big data to find novel associations between biological entities, pinpoint relevant biomarkers and build elaborate markers of disease and physiology. In doing so, multiomics integrates diverse omics data to find a coherently matching geno-pheno-envirotype relationship or association. In particular, the methods of the present disclosure are used for the quantitative correlation and spatial colocalization study between ISH and ICH. As mentioned above, immunohistochemistry (IHC) is an invaluable tool for the detection, localization, and quantification of antigens in preserved tissue for research and diagnostic purposes. In situ hybridization (ISH) is a unique molecular analysis method because it provides the precise microscopic localization of analytes like proteins, nucleic acids such as DNA, mRNA and microRNA in metaphase spreads and cell and tissue preparations. In particular, the methods of the present disclosure are used for cell type classification based on such multi-omics data. In a further embodiment, the methods of the present disclosure are used to study epigenetic modifications and chromatin structures. In particular, the methods of the present disclosure are used to identify spatial distribution, and cell-specific ISH signals (Cell segmentation).

In some further advantageous embodiments, the methods of the present disclosure may be used to determine the correlation of genetic aberrations and/or the copy number variations (CNV) to the corresponding proteins, wherein CNV is a phenomenon in which sections of the genome are repeated and the number of repeats in the genome varies between individuals.

### Experimental Data

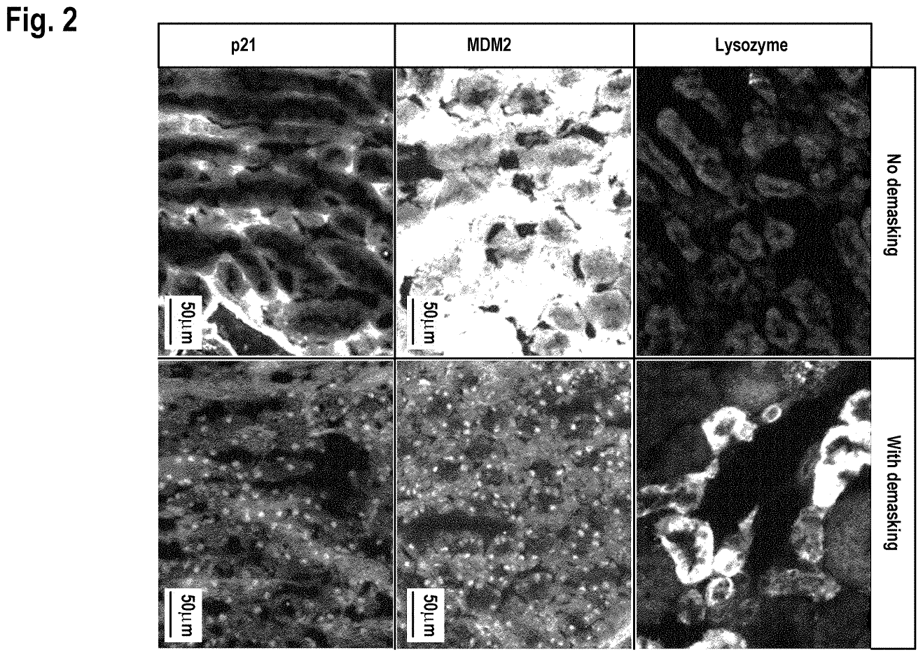

Cryosection of murine kidney tissues were sectioned using a cryostat (Leica Biosystems, CM1520) with a thickness of 10 μm. Tissues were dehydrated, rehydrated, fixed with 4% Parafomaldehyde for 30 min, and treated with the two-step demasking method. In particular, tissues were first incubated in a pH 6 buffer for 30 min at 70° C. The buffer was exchanged with a pH 9 buffer and tissues were incubated for 30 min at 70° C. Aferwards, tissues were probed for Lysozyme, MDM2, and p21. Rabbit primary antibody against lysozyme (BioLegend, #860001) and mouse primary antibody against MDM2 (Santa Cruz, sc-965) and p21 (Origene, AM09100PU-T) were incubated on the tissues. Alexa Flour 647 conjugated to goat anti-rabbit IgG (Abcam, ab150083) were added after the primary antibody incubation. Images were captured with ZEISS Celldiscoverer 7 using a Apochromat 5 X/0.35 objective and 2X Tubelens (see FIG. 2).

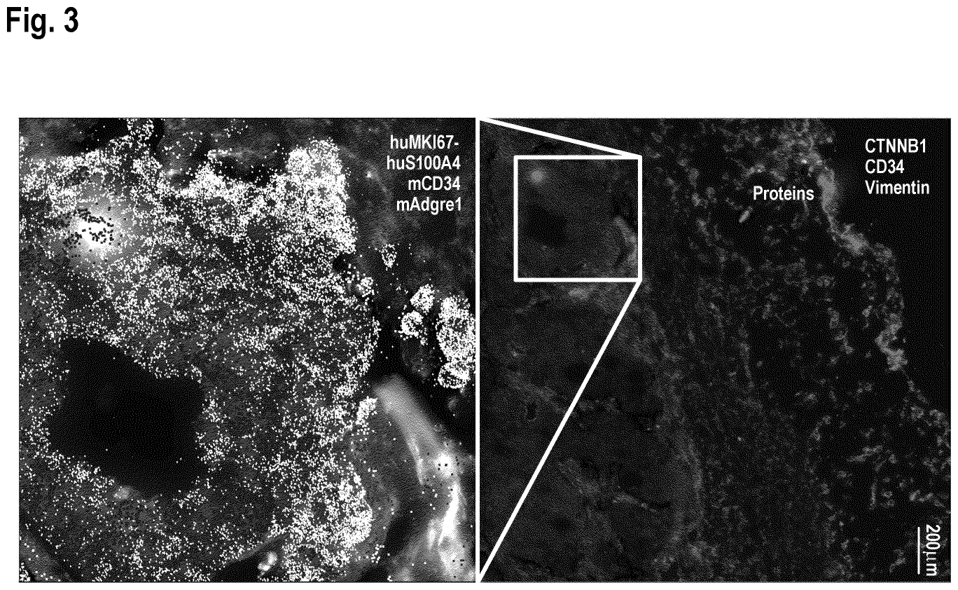

Cryosection of a tissue from a colorectal xenograft model was sectioned using a cryostat (Leica Biosystems, CM1520) with a thickness of 10 μm. Tissues were dehydrated, rehydrated, fixed with 4% paraformaldehyde. Molecular CartographyTM was performed, by which RNA transcripts of gene of interests were detected. After RNA transcripts detection, tissue was treated with the two-step demasking method. In particular, tissue was first incubated in a pH 6 buffer for 30 min at 70° C. The buffer was exchanged with a pH 9 buffer and tissues were incubated for 30 min at 70° C. Afterwards, tissue was probed for CTNNB1, CD34, and Vimentin. Rabbit primary antibody against CTNNB1 (Cell Signaling Technology, 8480S) and mouse primary antibody against CD34 (Invitrogen, #14-0341-82) and chicken primary antibody against Vimentin (BioLegend, #919101) were incubated on the tissue. Alexa Fluor® 594 conjugated goat anti-rat IgG (Abcam, ab150168), Alexa Flour® 546 conjugated to goat anti-rabbit IgG (Invitrogen, A11010), and Alexa Fluor® 488 conjugated goat anti-chicken IgY were added after the primary antibody incubation. Images were captured with ZEISS Celldiscoverer 7 using a Apochromat 50X/1.2 objective and 0.5X Tubelens (see FIG. 3).

1. A method of antigen retrieval of a fixed sample comprising the steps of:
a) incubating the fixed sample in a first antigen retrieval solution at a temperature between 50 to 75° C.;
b) transferring the fixed sample to a second antigen retrieval solution; and
c) incubating the fixed sample in the second antigen retrieval solution at a temperature between about 50 to 75° C.,
wherein the first antigen retrieval solution comprises a buffer solution having a pH range of between about 5 and 7 and the second antigen retrieval solution comprises a buffer solution having a pH range of between about 8 and 10; or
the first antigen retrieval solution comprises a buffer solution having a pH range of between about 8 and 10 and the second antigen retrieval solution comprises a buffer solution having a pH range of between about 5 and 7.

2. The method according to claim 1, wherein the fixed sample is a biological sample, preferably comprising biological tissue from human, animals, or plants, further preferably comprising biological cells and/or extracts and/or part of cells.

3. The method to any one of claims 1 to 2, wherein the cell is a prokaryotic cell or a eukaryotic cell, an animal cell, a plant cell, in particular a cell from vertebrate cells, in particular a mammalian cell, in particular a human cell.

4. The method to any one of claims 1 to 3, wherein the fixed sample is a frozen-fixed tissue sample, in particular a cryosection of a tissue.

5. The method to any one of claims 1 to 4, wherein the sample is fixed in crosslinking fixatives.

6. The method to any one of claims 1 to 5, wherein the sample is fixed in coagulant fixatives like alcohol, and acetone.

7. The method to any one of claims 1 to 6, wherein the sample is fixed in formaldehyde and embedded in paraffin (FFPE), in particular a cryosection of a formaldehyde-fixed biological sample.

8. The method to any one of claims 1 to 7, wherein the sample is a frozen fixed tissue sample and post-fixed with coagulant fixatives like alcohol or acetone and/or with crosslinking fixatives like formaldehyde.

9. The method to any one of claims 1 to 8, wherein the fixed sample is incubated in step a) for at least 0.5 h, in particular about 0.5 to 2 h.

10. The method to any one of claims 1 to 9, wherein the fixed tissue sample is incubated in step b) for at least 0.5 h, in particular for about 0.5 to 2 h

11. The method to any one of claims 1 to 10, wherein the fixed tissue sample is incubated in step a) for about 0.5 at a temperature of about 70° C.

12. The method to any one of claims 1 to 11, wherein the fixed tissue sample is incubated in step b) for about 0.5 at a temperature of about 70° C.

13. The method to any one of claims 1 to 12, wherein the buffer solution having a pH range of between about 5 and 7 comprises citric acid and the buffer solution having a pH range of between about 8 and 10 comprises tris(hydroxymethyl)methylamine (TRIS).

14. The method to any one of claims 1 to 13, wherein the buffer solution having a pH range between about 5 and about 7 comprises citric acid, potassium dihydrogen phosphate, boric acid, diethyl barbituric acid, piperazine-N,N′-bis(2-ethanesulfonic acid), dimethylarsinic acid, 2-(N-morpholino)ethanesulfonic acid, or a combination thereof.

15. The method to any one of claims 1 to 14, wherein the buffer solution having a pH range of between about 5 and about 7 comprises citric acid.

16. The method to any one of claims 1 to 15, wherein the buffer solution having a pH range of between about 8 and 10 comprises tris(hydroxymethyl)methylamine (TRIS), 2-(N-morpholino)ethanesulfonic acid (TAPS), N,N-bis(2-hydroxyethyl)glycine(Bicine), N-tris(hydroxymethyl)methylglycine (Tricine), 4-2-hydroxyethyl-I-piperazineethanesulfonic acid (HEPES), 2-{[tris(hydroxymethyl)methyl]amino}ethanesulfonic acid (TES), or a combination thereof.

17. The method to any one of claims 1 to 16, wherein the buffer solution having a pH range of between about 8 and 10 comprises TRIS and/or or boric acid.

18. The method according to one or more of the above claims, wherein the fixed tissue sample is a sectional portion of an organ or tissue, body fluid, a tissue or microarray.

19. The method according to one or more of the above claims, wherein the incubating steps with the first antigen retrieval solution and the second antigen retrieval solution comprises incubation in a heating device.

20. The method of claim 19, wherein the heating device is a pressure cooker, oven, stove, autoclaving, water bath, hot plate, microwave, steam heating, or combination thereof.

21. The method according to one or more of the above claims, further comprising the step of immunostaining of antigens.

22. The method of claim 21, wherein the immunostaining comprises sequential immunoperoxidase labeling and erasing.

23. The method according to one or more of the above claims, wherein the fixed tissue sample undergoes in-situ hybridization analysis.

24. The method according to one or more of the above claims, wherein the fixed tissue sample undergoes FISH or CISH analysis.

25. The method according to one or more of the above claims, wherein the fixed tissue sample undergoes spatial biology analysis like spatial transcriptomics and/or spatial proteomics.

26. The method according to one or more of the above claims, wherein before the antigen retrieval the fixed tissue sample is analyzed by an in-situ hybridization method and after the antigen retrieval the fixed tissue sample is analyzed by a immunohistochemical staining of the antigens.

27. The method according to one or more of the above claims, wherein after the antigen retrieval the fixed tissue sample is analyzed by an in-situ hybridization method and/or by an immunohistochemical staining of the antigens.

28. The method according to any one of claims 1 to 27, wherein the analytes are fixed in a permeabilized sample, such as a cell-containing sample.

29. The method to any one of claims 1 to 28, wherein one or more of the sample handling is automated.

30. The method according to any one of claims 1 to 5, wherein the antigen is a nucleic acid, preferably DNA, PNA or RNA, in particular mRNA.

31. The method according to any one of claims 1 to 5, wherein the antigen is a peptide, polypeptide or a protein.

32. The method according to any one of claims 1 to 31 wherein all steps are automated, in particular by using a robotic system.

33. The method according to any one of claims 1 to 32, wherein all steps are performed in a fluidic system.

34. The method according to any of claims 1 to 33, wherein the antigen is detected by:
(a) Imaging at least a portion of the sample; and/or
(b) Using an optical imaging technique; and/or
(c) Using a fluorescence imaging technique; and/or
(d) Multi-color fluorescence imaging technique; and/or
(e) Super-resolution fluorescence imaging technique.

35. A method according to any of the proceeding items for the use in an application selected from the group consisting of identify regions of interest, for image analysis, develop cell segmentation algorithms using immunofluorescence images, to aid the segmentation of cells in a sample, to establish Al-based image analysis algorithm using immunofluorescence images to identify disorders, feature extraction like nucleus morphology, signal homogeneity, signal intensity and/or centroids, to determine expression of proteins and genes of interest via semi-quantitative approaches, diagnosis and prognosis applications, determination of the distribution of a target protein in the of health and disease organ, tissue and/or cell, to predict the prognosis of diseases by using disease-related prognostic and/or predictive biomarkers, to provide disease diagnosis e.g. tumor of unknow origin, neurodegenerative disorders, muscle disorders, brain trauma, infectious diseases, to predict a therapeutic response, subclassification of diseases, examine gene expression and to study gene regulation in normal and pathological tissues, morphological studies, recognition of tissue structures and/or cell type classifications, multiomics studies, quantitative correlation and spatial colocalization study between ISH and ICH, study epigenetic modifications and chromatin structures, identify spatial distribution, and cell-specific ISH signals (Cell segmentation) and determine the correlation of genetic aberrations and/or the copy number variations (CNV) to the corresponding proteins.
